(19) United States
(12) Patent Application Publication
(10) Pub. No.: US 2010/0093804 A1
(43) Pub. Date: Apr. 15, 2010

(54) NOVEL CRYSTALLINE FORM OF LANSOPRAZOLE

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, Andhrapradesh (IN)

(21) Appl. No.: 11/993,960

(22) PCT Filed: Dec. 7, 2006
(86) PCT No.: PCT/IN06/00482

§ 371 Date: Dec. 26, 2007

(51) Int. Cl. A61K 31/435; C07D 401/12; A61P 1/00

(52) U.S. Cl. 514/338; 546/273.7

(57) ABSTRACT

The present invention relates to a novel and stable crystalline polymorph of lansoprazole, a process for its preparation and to a pharmaceutical composition comprising it. Thus, for example, lansoprazole crude is dissolved in methanol at 20-30° C. followed by stirring and the solution is cooled to 0-10° C. The resulting solution is stirred for 1 hour to 1 hour 30 minutes at 0-10° C., the solid is filtered and then dried to give lansoprazole crystalline form III.

### FIELD OF THE INVENTION

The present invention relates to a novel and stable crystalline polymorph of lansoprazole, process for its preparation and to a pharmaceutical composition comprising it.

### BACKGROUND OF THE INVENTION

European Patent Nos. 0174726 and 0302720 disclosed 2-(2-pyridylmethylsulfinyl)-benzimidazole derivatives and their pharmaceutically acceptable salts, process for their preparation and therapeutic use thereof. These compounds are anti-ulcer agents, and useful for prophylaxis and therapy of digestive ulcers (e.g. gastric ulcer, duodenal ulcer) and gastritis. Among these compounds, lansoprazole, chemically, 2-[[[3-Methyl-4-(2,2,2-trifluoro-ethoxy)-2-pyridinyl]methyl]sulfinyl]-1H-benzimidazole is a well-known gastric acid secretion inhibitor and used in the treatment of gastric and duodenal ulcers. Lansoprazole is represented by the following structure:

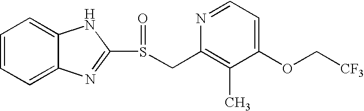

Lansoprazole can exist in different polymorphic forms, which differ from each other in terms of stability, physical properties, spectral data and methods of preparation.

The European Patent No. 0302720 makes no reference to the existence of specific polymorphic forms of lansoprazole. In this patent, it is disclosed that the compound is isolated according to conventional techniques; more precisely, according to the embodiments exemplified, crude lansoprazole (obtained by oxidation of 2-[[[3-Methyl-4-(2,2,2-trifluoro-ethoxy)-2-pyridinyl]methyl]thio]-1H-benzimidazole with hydrogen peroxide in the presence of catalytic amounts of vanadium pentoxide) is purified by crystallization from aqueous ethanol (up to 10% water content).

PCT Patent publication No. WO 98/21201 discloses solvent-free crystalline forms of lansoprazole and methods for their preparation thereof.

PCT Patent Publication No. WO 00/78729 A1 described two crystalline forms of lansoprazole (form I and form II), characterizes them by powder X-ray diffraction, infra-red spectroscopy and differential scanning calorimetry (DSC). The publication further described that the synthetic procedure described and exemplified in the European Patent No. 0302720 produces the lansoprazole crystalline form II, characterized by an X-ray powder diffraction pattern having peaks expressed as 2θ at about 5.16, 5.54, 6.77, 10.44, 11.17, 15.76, 16.73, 17.81, 18.50, 21.13, 22.67 and 27.68 degrees.

The PCT publication further taught that the lansoprazole crystalline form II is stable at temperatures below 0° C. However, after some period of sample storage at higher temperatures, form II is converted to stable crystalline form I of lansoprazole (characterized by an X-ray powder diffraction pattern having peaks expressed as 2θ at about 5.56, 6.85, 11.24, 14.05, 16.73, 17.37, 18.47, 22.16, 22.74, 23.31, 24.79, 25.63, 27.61 and 31.09 degrees). The PCT publication further disclosed a method of preparation of lansoprazole in the crystalline form I consists in that a crude lansoprazole is subjected to crystallization from aqueous ethanol containing up to 10% water, at temperature from 20° C. to 60° C., preferably 55-60° C., and then the resulting lansoprazole is crystallized from acetone and isolated by a known method.

Eur. J. Pharm. Sci. volume 4, page 182 (1996 Supp.) (Kotar et al.) described two polymorphs of lansoprazole, designated as crystalline lansoprazole form A and form B. According to Kotar, DSC curve of the form A showed only an endothermic peak at 180° C., while the form B showed one exothermic peak at 102° C. and one endothermic peak at 180° C. In fact, crystalline lansoprazole form B is unstable and can undergo a solid-solid transition to form crystalline lansoprazole form A. Kotar provides no XRD data for crystalline lansoprazole forms A and B, and fails to disclose processes for preparing these crystalline forms.

Farm. Vest. volume 50, page 347 (1999) described an amorphous form of lansoprazole prepared by spray drying method.

PCT Patent Publication No. WO 03/082857 A2 describes three crystalline forms of lansoprazole (form D, form E and form F), and processes for their preparation thereof. The publication also teaches processes for preparing crystalline lansoprazole form A, comprising the steps of: a) preparing a solution of lansoprazole in a solvent selected from the group consisting of methanol, n-butanol, acetone, methylethylketone, ethyl acetate, dimethylsulfoxide, dimethyl formamide and their mixtures optionally with water; and b) isolating crystalline lansoprazole form A.

As per the purification process exemplified in the PCT Patent Publication No. WO 03/082857 A2, crystalline lansoprazole form A is purified by dissolving crystalline lansoprazole form A in methanol, heating the, solution to reflux, cooling the methanol solution to ambient temperature to induce precipitation of lansoprazole and then collecting the crystalline lansoprazole form A. The publication provides no XRD data for crystalline lansoprazole form A.

All patents and patent publications cited herein are incorporated by reference in their entirety.

We have discovered a novel and highly stable crystalline form of lansoprazole, designated as form III, which differ from each of the prior art forms, in their stability, in their spectral characteristics and in their method of preparation. The novel crystalline lansoprazole form III is stable over the time, can be reproducible consistently and has good flow properties and so, the novel crystalline form III is suitable for formulating lansoprazole.

The object of the present invention is to provide a stable and consistently reproducible novel crystalline form of lansoprazole, process for preparing it and a pharmaceutical composition comprising it.

### DETAILED DESCRIPTION OF THE INVENTION

According to one aspect of the present invention, there is provided a novel crystalline form of lansoprazole, designated as lansoprazole form III and typical X-ray powder diffraction spectrum of lansoprazole form III is depicted in FIG. 1.

Lansoprazole form III is characterized by an X-ray powder diffraction pattern having peaks expressed as 2θ angle positions at about 5.6, 11.3, 12.7, 14.2, 16.9, 17.5, 18.6, 22.3, 23.4, 24.9, 25.6, 25.8, 27.7, 30.2 and 31.2±0.1 degrees.

According to another aspect of the present invention, there is provided a process for the preparation of crystalline lansoprazole form III, which comprises:
a) dissolving lansoprazole in methanol at about 15-30° C.;
b) stirring the solution formed in step (a) at about 15-30° C.; and
c) crystallizing lansoprazole crystalline form III from the solution obtained in step (b).

The solution in step (b) is preferably stirred at least for about 30 minutes, more preferably stirred at least for about 1 hour and still more preferably stirred for about 1 hour to 3 hours.

Crystallization in step (c) is carried out by cooling the solution obtained in step (b) to below 10° C., preferably to 0-10° C. and optionally seeding with lansoprazole crystalline form III. The crystals of lansoprazole form III formed in step (c) are collected by filtration or centrifugation.

Lansoprazole used as starting material may be obtained by processes described in the art, for example by the processes described in the European Patent Nos. 0174726 and 0302720.

The novel crystalline form can be produced in a consistently reproducible manner by simple procedures. The novel crystalline form is obtained polymorphically pure with no or less contamination with other crystalline forms.

According to another aspect of the present invention there is provided a pharmaceutical composition comprising lansoprazole crystalline form III and a pharmaceutically acceptable excipient.

Preferable pharmaceutical composition of lansoprazole crystalline form III is a solid oral dosage form.

### BRIEF DESCRIPTION OF THE DRAWING

FIG. 1 is a powder X-ray diffraction spectrum of crystalline lansoprazole form III.

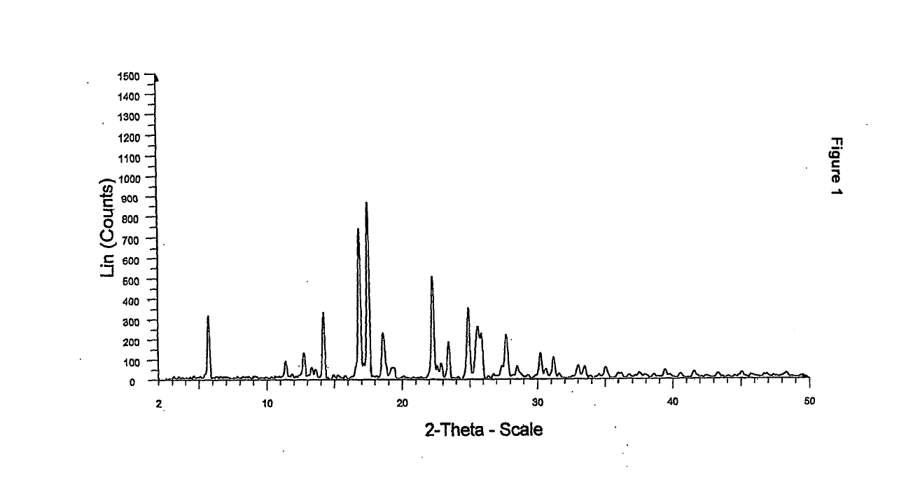

X-ray powder diffraction spectrum was measured on a Brucker axs D8 advance X-ray powder diffractometer having a copper-Kα radiation. Approximately 1 gm of the sample was gently flattened on a sample holder and scanned from 2 to 50 degrees two theta, at 0.03 degrees two-theta per step and a step time of 0.5 seconds. The sample was simply placed on the sample holder. The sample was rotated at 30rpm at a voltage of 40 KV and current 35 mA.

The following example is given for the purpose of illustrating the present invention and should not be considered as limitations on the scope or spirit of the invention.

### EXAMPLE

Lansoprazole crude (10 gm) is dissolved in methanol (50 ml) at 20-30° C., stirred for 1 hour to 1 hour 30 minutes at 20-30° C. and then the solution is cooled to 0-10° C. The resulting solution is stirred for 1 hour to 1 hour 30 minutes at 0-10° C., filtered the solid, washed with methanol (15 ml) and then dried the material at 40-50° C. to give 9.6 gm of lansoprazole crystalline form

We claim:

1. A crystalline lansoprazole form III, characterized by an X-ray powder diffraction pattern having peaks expressed as 2θ angle positions at about 5.6, 11.3, 12.7, 14.2, 16.9, 17.5, 18.6, 22.3, 23.4, 24.9, 25.6, 25.8, 27.7, 30.2 and 31.2±0.1 degrees.

2. A process for preparation of crystalline lansoprazole form III as defined in claim 1, which comprises:
a) dissolving lansoprazole in methanol at about 15-30° C.;
b) stirring the solution formed in step (a) at about 15-30° C.; and
c) crystallizing lansoprazole crystalline form Ill from the solution obtained in step (b).

3. The process as claimed in claim 2, wherein the solution in step (b) is stirred at least for about 30 minutes.

4. The process as claimed in claim 3, wherein the solution is stirred at least for about 1 hour.

5. The process as claimed in claim 4, wherein the solution is stirred for about 1 hour to 3 hours.

6. The process as claimed in claim 2, wherein the crystallization in step (c) is carried out by cooling the solution obtained in step (b) to below 10° C.

7. The process as claimed in claim 6, wherein the solution is cooled to 0-10° C.

8. The process as claimed in claim 2, wherein the crystals of lansoprazole form III formed in step (c) are collected by filtration or centrifugation.

9. A pharmaceutical composition comprising lansoprazole crystalline form III of claim 1 and a pharmaceutically acceptable excipient.

10. The pharmaceutical composition as claimed in claim 9, wherein the pharmaceutical composition of lansoprazole crystalline form III is a solid oral dosage form.
